(19) United States
(12) Patent Application Publication
(10) Pub. No.: US 2022/0370339 A1
(43) Pub. Date: Nov. 24, 2022

(54) SCALP CLEANSING WIPES AND TREATMENT FORMULATION

(71) Applicant: LaShondria Camp, Lewisville, TX (US)

(72) Inventor: LaShondria Camp, Lewisville, TX (US)

(21) Appl. No.: 16/435,497

(22) Filed: Jun. 9, 2019

(51) Int. Cl. A61K 8/9789; A61K 8/9794; A61K 8/67; A61K 8/65; A61K 8/34; A61K 8/365; A61K 8/27; A61K 8/9728; A61Q 19/10; A61K 8/02

(52) U.S. Cl. CPC A61K 8/9789; A61K 8/9794; A61K 8/676; A61K 8/673; A61K 8/65; A61K 8/347; A61K 8/365; A61K 8/27; A61K 8/9728; A61Q 19/10; A61K 8/0208; A61K 2800/524

(57) ABSTRACT

A cleansing substrate comprising a formulation selected from the group consisting of distilled water, lemon juice, liquid aloe, aloe gel, liposomal Vitamin C, Zing, B5, B6 Keratin, Grapefruit seed oil, Vitamin E oil, Critic acid, and menthol. Wherein the cleansing substrate is used to clean and treat the hair and scalp of a person.

### CROSS-REFERENCE TO RELATED APPLICATIONS

This application the benefit U.S. Provisional Application No. 62/682,749, filed Jun. 8, 2018, and U.S. Provisional Application No. 62/857,806, filed Jun. 5, 2019.

### BACKGROUND

Protective hairstyles are common to promote healthy hair growth, by tucking away the most fragile part of the hair strands at their ends, while styling the hair into a variety of forms. Among the most common forms are braids and twists. While protective hairstyles are great for promoting hair growth, they are more difficult to clean with the typical shampoo and water, as would be done with other hairstyles. As such, maintaining a clean scalp becomes more difficult, which leads to undesirable outcomes, like an itchy scalp and dandruff.

Personal hygiene products currently being marketed and sold as hair cleansers, scalp treatment, shampoos and conditioners are typically in liquid form. Instructions provided with the cleanser instruct the consumer to apply the product directly to damp hair, then lather and rinse, which is not feasible for hair in protective styles. Alternatively, products such as “dry” shampoos have been developed to address not wetting styled hair. However, using dry shampoo results in dry hair and itchy scalp. Cleansing the head and scalp of styled hair remains a challenge.

The need exists for a product that can allow one to clean their scalp while it is in a protective style, without drying their hair or scalp in the process.

### BRIEF SUMMARY

The current apparatus addresses many problems consumers encounter while cleaning and treating their hair and scalp while still maintaining their protective hairstyle. Hair and Scalp Cleansing Wipes can be used as an adjunct or between regular shampooing and conditioning to keep the scalp and hair free of excess oils and debris when regular washing may not be feasible while wearing protective hairstyles.

The system provides a hair treating method using the sheet-like hair care product. The method includes the steps of (i) optionally picking up the sheet-like hair care product, which is infused with the liquid cosmetic composition, from the packaging container through the access opening, (ii) spreading out the sheet-like hair care product on the palmer side of a user's hand in such a manner that a part of the sheet-like hair care product hangs over the fingertips, and (iii) moving the user's hand with the sheet-like hair care product thereon from the roots to the tips of hair strands to treat the hair strands.

The system also provides a method for cleaning the scalp using the sheet-like hair care product of the invention by applying the hand holding the sheet-like hair care product to the scalp and rubbing the fingertips against the scalp via the sheet-like hair care product.

In one aspect of the present apparatus, there is provided a cleansing formulation comprising ingredients from the list in Table 1 and a substrate to remove soiled substance, without drying out the scalp, to relieve itchy and or dry scalp.

TABLE 1

| | |
|---|---|
| a. | Purified Water (H₂0) |
| b. | Aloe Vera |
| c. | Lemon juice |
| d. | Liposomal Ascorbic Acid |
| e. | Cobalamin |
| f. | Zinc Oxide |
| g. | Ricinus Communis Seed Oil |
| h. | Tocopheryl Acetate |
| i. | Citrus Grandis Extract |
| j. | Rosmarinus Officinalis Leaf Oil |
| k. | Peppermint |
| l. | Biotin |
| m. | B5 |
| n. | B6 |
| o. | Keratin |
| p. | Collagen |
| q. | Cayenne |
| r. | Eleuthero |
| s. | Stinging Nettle |
| t. | Horsetail |
| u. | Reishi |
| v. | Grapefruit seed oil |
| w. | Vitamin E oil |
| x. | Critic acid |
| y. | Menthol |
| z. | Phenoxyethanol |
| aa. | Butylcarbarnate |

In one embodiment, the cleansing formulation comprises of a mild strength for sensitive skin. In another embodiment, the cleansing formulation comprises of a normal strength formula. In a third embodiment, the cleansing formulation comprises of a composition to allow for volumizing and moisturizing. In another embodiment, the cleansing formula comprises an extra strength formula for a deep clean. In yet another embodiment, the cleansing formulation will be delivered via a natural cotton conduit designed to be biodegradable and disposable.

Unless specifically noted otherwise herein, the definitions of the terms used are standard definitions used in the art of manufacturing shampoo, such as no-rinse shampoo. Exemplary embodiments, aspects and variations are illustrative in the figures and drawings, and it is intended that the embodiments, aspects and variations, and the figures and drawings disclosed herein are to be considered illustrative and not limiting.

In one aspect of the present application, there is provided cleansing substrate formulation comprising one or more ingredients of Table 1, wherein the substrate is used to clean and treat hair and scalp.

In one embodiment, the formulation comprises different portions of the ingredients of Table 1 to make a mild formula for sensitive skins. In another embodiment, the formulation comprises different portions of the ingredients of Table 1 to make an extra strength formula for a deep clean.

### BRIEF DESCRIPTION OF THE DRAWINGS

FIG. 1 is perspective view of a wipe, a chart displaying the ingredients contained in the wipe, and a person.

FIG. 2 is a chart displaying the active ingredients contained in the wipe.

FIG. 3 is a flow chart illustrating the method of applying a wipe to a scalp.

### DETAILED DESCRIPTION

The following detailed description illustrates embodiments of the present disclosure. These embodiments are described in sufficient detail to enable a person of ordinary skill in the art to practice these embodiments without undue experimentation. It should be understood, however, that the embodiments and examples described herein are given by way of illustration only, and not by way of limitation. Various substitutions, modifications, additions, and rearrangements may be made that remain potential applications of the disclosed techniques. Therefore, the description that follows is not to be taken as limiting on the scope of the appended claims. In particular, an element associated with a particular embodiment should not be limited to association with that particular embodiment but should be assumed to be capable of association with any embodiment discussed herein.

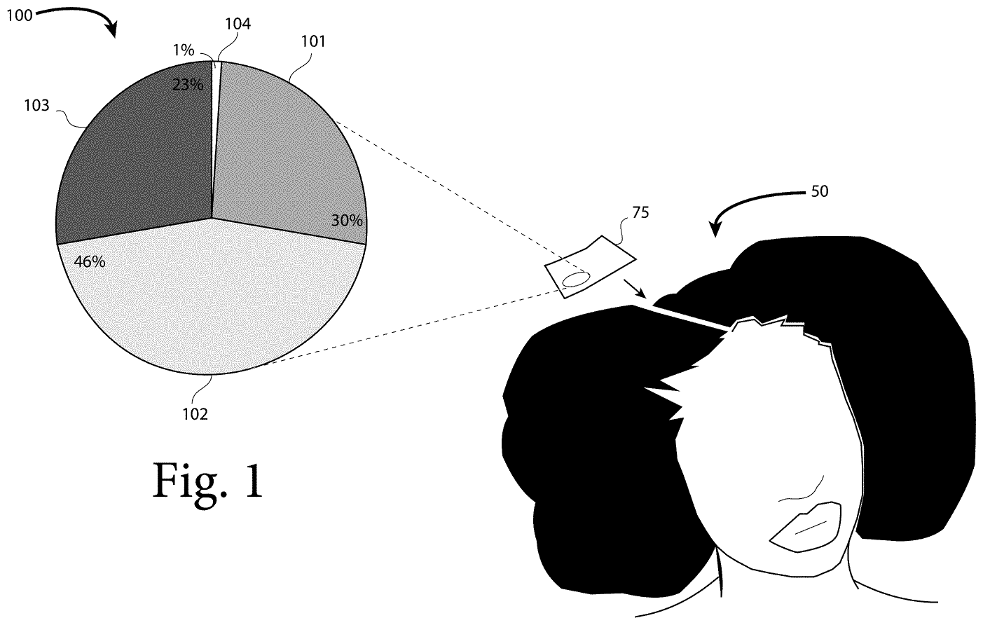

FIG. 1 is an embodiment of a person 50 using a wipe 75 saturated with a solution 100 of distilled water 101, lemon juice 102, liquid aloe 103, and several active ingredients, fragrance, vitamins, and proteins 104 formulated to improve hair health. It also shows the approximate percentages of each component of the solution as 30%, 46%, 23%, and 1% respectively.

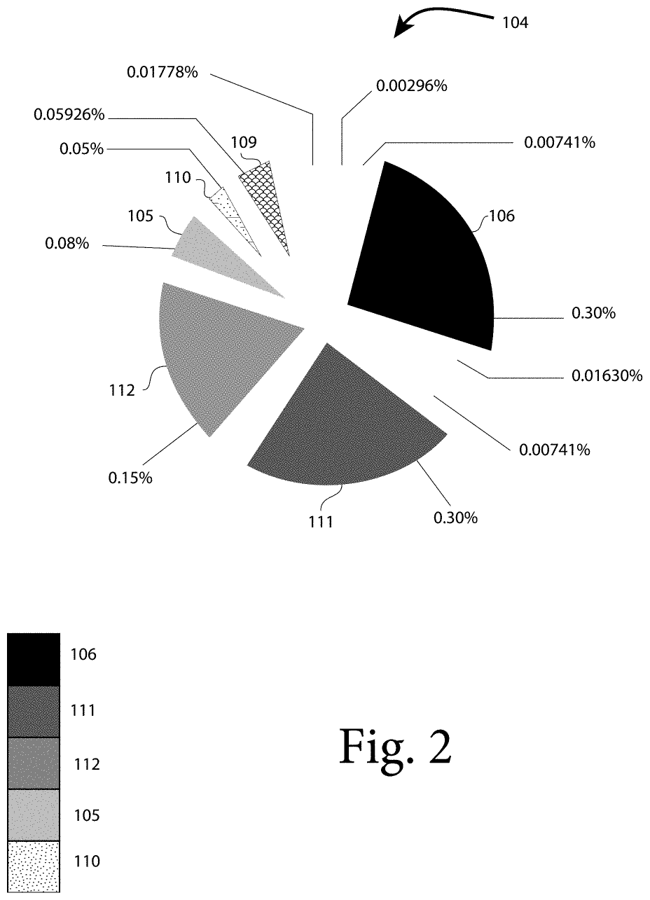

FIG. 2 shows the breakdown of all the active ingredients that makeup the ingredients that are in 104. These ingredients include Liposomal Vitamin C 105, Ionic Zinc 106, Biotin 108, B5 109, B6 110, Keratin 111, organic herbs liquid 112, grape seed 113, Vitamin E 114, Citric Acid 115, Methanol 116, and Preservative 117. With regards to the organic herbs liquid 112, in this embodiment it consists of cayenne, eleuthero, stinging nettle, horsetail, and reishi. It also shows the approximate percentages of each component of the solution as: 105—0.00741%, 106—0.30%, 108—0.00889%, 109—0.0163%, 110—0.00741%, 111—0.30%, 112—0.15%, 113—0.08%, 114—0.05%, 115—0.05926%, 116—0.01778%, and 117—0.00296%.

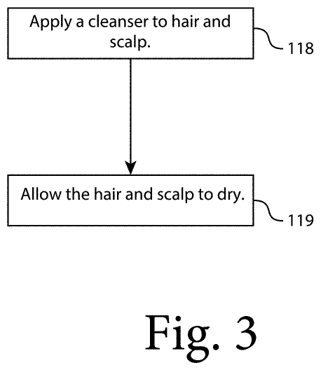

FIG. 3 shows the process or method of using said apparatus to cleanse the scalp and hair. Once the product has been removed from its packaging the user should apply the sheet to the scalp and hair throughout to ensure the product is distributed evenly and thoroughly. Once the product has been applied properly it should be allowed to air dry. As the product is being used it removes dirt, dry skin, and oily buildup that are all unhealthy for the human hair and scalp. The invention replaces these harmful substances with healthy oils and fragrances leaving the hair smelling fresh and renewed.

The operations of the flow diagrams are described with references to the systems/apparatus shown in the block diagrams. However, it should be understood that the operations of the flow diagrams could be performed by embodiments of systems and apparatus other than those discussed with reference to the block diagrams, and embodiments discussed with reference to the systems/apparatus could perform operations different than those discussed with reference to the flow diagrams.

The text above describes one or more specific embodiments of a broader invention. The invention also is carried out in a variety of alternate embodiments and thus is not limited to those described here. The foregoing description of an embodiment of the invention has been presented for the purposes of illustration and description. It is not intended to be exhaustive or to limit the invention to the precise form disclosed. Alternatives ingredients and methods may be used to enhance or substitute those listed above and may be used in different percentages and orders. Many modifications and variations are possible in light of the above teaching. It is intended that the scope of the invention be limited not by this detailed description, but rather by the claims appended hereto.

What is claimed is:

1. A cleansing substrate comprising:
a formulation selected from the group consisting of distilled water, lemon juice, aloe vera, liposomal Vitamin C, Zing, B5, B6 Keratin, Grapefruit seed oil, vitamin E oil, Critic acid, and menthol;
wherein the cleansing substrate is used to clean and treat the hair and scalp of a person.

2. The cleansing substrate of claim 1 wherein the cleansing substrates includes nonwoven material.

3. The cleansing substrate of claim 2 wherein the nonwoven material includes biodegradable material.

4. The cleansing substrate of claim 1 wherein the formulation further includes biotin.

5. The cleansing substrate of claim 1 wherein the formulation further includes collagen.

6. The cleansing substrate of claim 1 wherein the formulation is further selected from the group cayenne, eleuthero, stinging nettle, horsetail, and reishi.

7. The cleansing substrate of claim 1 wherein the formulation further includes phenoxyethanol and butyclarbarnate.

8. The cleansing substrate of claim 1 wherein the formulation further includes a preservative.

9. The cleansing substrate of claim 8 wherein the preservative includes butylcarbarnate.

10. The cleansing substrate of claim 8 wherein the preservative includes phenoxyethanol.

11. A method comprising:
applying a cleansing substrate to a hair and a scalp of a person; and
allowing the hair and scalp to dry;

12. The method of claim 11 wherein the cleansing substrates includes nonwoven material.

13. The method of claim 12 wherein the nonwoven material includes biodegradable material.

14. The method of claim 11 wherein the formulation further includes biotin.

15. The method of claim 11 wherein the formulation further includes collagen.

16. The method of claim 11 wherein the formulation is further selected from the group cayenne, eleuthero, stinging nettle, horsetail, and reishi.

17. The method of claim 11 wherein the formulation further includes phenoxyethanol and butyclarbarnate.

18. The method of claim 11 wherein the formulation further includes a preservative.

19. The method of claim 18 wherein the preservative includes butylcarbarnate.

20. The method of claim 18 wherein the preservative includes phenoxyethanol.
